(19) United States
(12) Patent Application Publication
(10) Pub. No.: US 2009/0069804 A1
(43) Pub. Date: Mar. 12, 2009

(54) Apparatus for efficient power delivery

(76) Inventor: Jeffrey L. Jensen, Boulder, CO (US)

Correspondence Address: Tyco Healthcare Group LP, 60 MIDDLETOWN AVENUE, NORTH HAVEN, CT, 06473, US

(21) Appl. No.: 11/900,474

(22) Filed: Sep. 12, 2007

(51) Int. Cl. A61B 18/12

(52) U.S. Cl. 606/41

(57) ABSTRACT

A delivery apparatus for supporting an electrosurgical instrument and for connecting the electrosurgical instrument to an electrosurgical energy source is provided. The delivery apparatus includes a selectively positionable housing including a plurality of rigid segments and a joint member interposed between each adjacent rigid member; and a transmission medium extending through at least one of the segments of the housing. The transmission medium is configured to transmit electrosurgical energy between the electrosurgical energy source and the electrosurgical instrument. In use, the housing is movable between a first position wherein an electrosurgical instrument supported on an end of the housing is remote from an operative site and a second position wherein the electrosurgical instrument supported on the end of the housing is proximate to the operative site.

### BACKGROUND

1. Technical Field

The present disclosure relates to medical/surgical therapeutic energy delivery procedures and apparatus and, more particularly, to an apparatus for efficient power delivery in electrosurgical energy delivery procedures and methods of efficiently delivering said electrosurgical energy to an electrosurgical treatment device.

2. Background of Related Art

In the treatment of diseases, such as cancer, certain types of cancer cells have been found to denature at elevated temperatures (which are slightly lower than temperatures normally injurious to healthy cells). These types of treatments, known generally as hyperthermia therapy, typically utilize electromagnetic radiation to heat diseased cells to temperatures above 41° C. while maintaining adjacent healthy cells at lower temperatures where irreversible cell destruction will not occur. Other procedures utilizing electromagnetic radiation to heat tissue also include ablation and coagulation of the tissue.

Such electrosurgical procedures benefit from efficient transfer of electrosurgical energy from a point of power generation, e.g., a generator, to the point of application or to the treatment device. The overall efficiency of the electrosurgical energy transfer is dependent on the transmission path between the point of power generation and the point of application. The transmission path may include an elongated transmission medium or conductor, e.g., a rigid, semi-rigid or flexible coaxial cable, waveguide or other suitable conductor, one or more connectors, a transmission line of the electrosurgical energy delivery device and the electrosurgical energy delivery or treatment device itself, i.e., a microwave antenna, mono-polar or bi-polar RF energy delivery device or other type of applicator for power delivery.

Flexible conductors inherently increase the power dissipated or lost along the transmission medium, due to the materials and dimensional configurations of their constructions. Non-flexible conductors (e.g., rigid, semi-rigid or waveguide transmission conductors), while potentially dissipating less energy along the transmission path as compared to flexible conductors, generally limit the ability to freely position and maintain the position of the electrosurgical energy delivery device as well as limit the movement and position of operators in the vicinity thereof.

### SUMMARY

The present disclosure relates an apparatus for efficient power delivery in electrosurgical energy delivery procedures and methods of efficiently delivering said electrosurgical energy to an electrosurgical treatment device.

According to an aspect of the present disclosure, a delivery apparatus for supporting an electrosurgical instrument and for connecting the electrosurgical instrument to an electrosurgical energy source is provided. The delivery apparatus includes a selectively positionable housing including a plurality of rigid segments and a joint member interposed between each adjacent rigid member; and a transmission medium extending through at least one of the segments of the housing. The transmission medium is configured to transmit electrosurgical energy between the electrosurgical energy source and the electrosurgical instrument. In use, the housing is movable between a first position wherein an electrosurgical instrument supported on an end of the housing is remote from an operative site and a second position wherein the electrosurgical instrument supported on the end of the housing is proximate to the operative site.

According to another aspect of the present disclosure, a system for maneuvering an electrosurgical instrument to/from an operative site and for connecting the electrosurgical instrument to an electrosurgical energy source is provided. The system includes an electrosurgical energy source; an electrosurgical instrument; and a delivery apparatus supported on and electrically coupled to the electrosurgical energy source. The delivery apparatus includes a selectively positionable housing including a plurality of rigid segments and a joint member interposed between each adjacent rigid member, the housing being configured to support the electrosurgical instrument at a free end thereof; and a transmission medium extending between the electrosurgical energy source and the electrosurgical instrument, through at least one of the segments of the housing, wherein the transmission medium is configured to transmit electrosurgical energy between the electrosurgical energy source and the electrosurgical instrument. In use, the housing is movable between a first position wherein the electrosurgical instrument supported on the free end of the housing is remote from the operative site and a second position wherein the surgical instrument supported on the free end of the housing is proximate to the operative site.

The transmission medium may be continuous and flexible along at least a portion of its length. The transmission medium may include a plurality of segments electromechanically joined to one another. Each segment of the housing may support at least one segment of the transmission medium.

The system may further include an electromechanical coupling inter-connecting adjacent segments of the transmission medium. Each electromechanical coupling may be configured to permit adjacent segments of the transmission medium to pivot off axis relative to one another and/or rotate axially relative to one another.

The transmission medium may be a relatively flexible coaxial cable, a semi-rigid coaxial cable, a fully rigid coaxial cable, a fully rigid coaxial cable with an air dielectric, and/or a wave-guide.

The housing may prevent bending of the segments of the transmission medium along their individual lengths.

According to yet another aspect of the present disclosure, a method of maneuvering an electrosurgical instrument to/from a surgical site, wherein the electrosurgical instrument is electrically connected to an electrosurgical energy source is provided. The method includes the steps of providing a delivery apparatus supported on and electrically coupled to the electrosurgical energy source; supporting a transmission medium on a housing of the delivery apparatus; supporting the electrosurgical instrument on a free end of the housing and electrically connecting the electrosurgical instrument to the transmission medium; manipulating the housing to move the electrosurgical instrument to a first position to perform an electrosurgical procedure; and manipulating the housing to move the electrosurgical instrument to a second position upon completion of the electrosurgical procedure.

The method may further include the step of minimizing a loss of energy transmitted from the electrosurgical energy source to the electrosurgical instrument. The method may further include the step of minimizing bending of the transmission medium between the electrosurgical energy source and the electrosurgical instrument.

### BRIEF DESCRIPTION OF THE DRAWINGS

Embodiments of the presently disclosed electrosurgical energy delivery apparatus are disclosed herein with reference to the drawings, wherein:

FIG. 1 is a schematic illustration of a system for supplying electrosurgical energy for therapeutic energy therapy, including the electrosurgical energy delivery apparatus according to an embodiment of the present disclosure;

FIG. 2A is a schematic illustration of exemplary energy delivery devices configured for connection to the electrosurgical energy delivery apparatus of FIG. 1;

FIG. 2B is a schematic illustration of an exemplary energy delivery device configured for connection to the electrosurgical energy delivery apparatus of FIG. 1;

FIG. 3 is a schematic, side elevational view of the electrosurgical energy delivery apparatus of FIG. 1; and

FIG. 4 is a top, plan view of the electrosurgical energy delivery apparatus of FIGS. 1 and 2.

### DETAILED DESCRIPTION

Embodiments of the presently disclosed electrosurgical energy delivery apparatus will now be described in detail with reference to the drawing figures wherein like reference numerals identify similar or identical elements. As used herein and as is traditional, the term “distal” refers to the portion of the device or instrument that is furthest from the user and the term “proximal” refers to the portion of the device or instrument that is closest to the user. In addition, terms such as “above”, “below”, “forward”, “rearward”, etc. refer to the orientation of the figures or the direction of components and are simply used for convenience of description.

One component that affects the efficiency of electrosurgical energy delivery between a point of power or energy generation (e.g., generator) and patient tissue and/or an energy delivery device is the loss of energy or energy dissipation along a transmission medium. The transmission medium may contain one or more components between the output connector on the electrosurgical generator and the input connector on the electrosurgical energy delivery device.

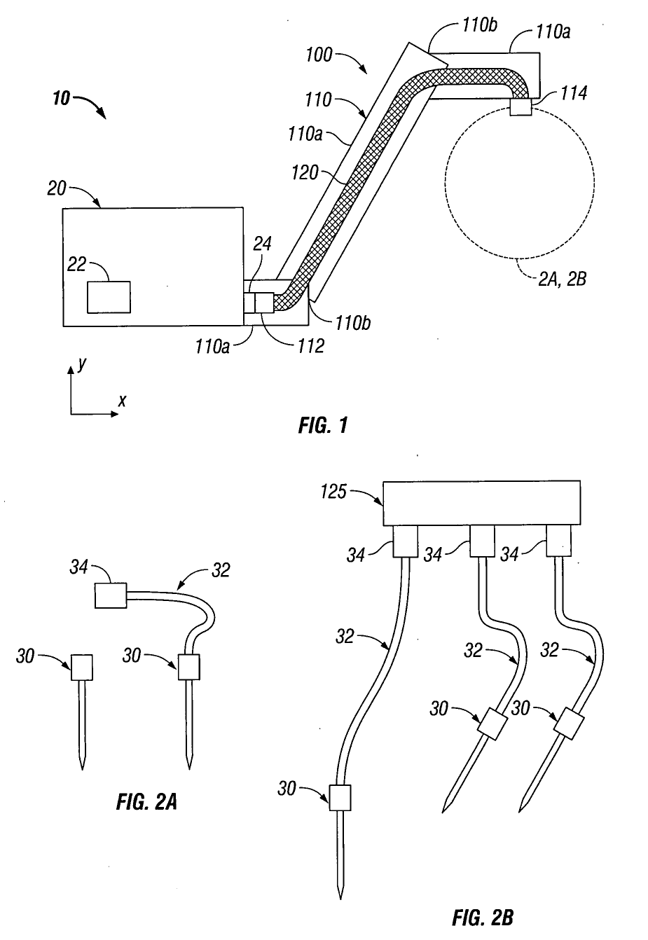

Referring now to FIG. 1, a system for supplying electrosurgical energy (e.g., microwave energy) for therapeutic energy therapy (e.g., microwave energy treatment), according to an embodiment of the present disclosure, is shown as 10. System 10 includes an electrosurgical generator 20 having a controller 22 for controlling the operation of electrosurgical generator 20, at least one electrosurgical delivery instrument or device 30 (see FIGS. 2A and 2B), and an electrosurgical energy delivery apparatus 100, according to an embodiment of the present disclosure, coupled between at least one power output 24 on the electrosurgical generator 20 and the at least one device 30.

In the illustrated embodiment, delivery apparatus 100 includes a jointed, articulating arm, in the form of a rig, boom, armature or housing 110 coupled to electrosurgical generator 20. Housing 110 is configured to house at least a portion of a transmission medium 120 therein. Delivery apparatus 100 includes a first connector 112 electrically connected to a first end of transmission medium 120, proximate a proximal end of housing 110, and configured for connection to a complementary connector 24 provided on or in electrosurgical generator 20. Delivery apparatus 100 further includes at least a second connector 114 electrically connected to a second end of transmission medium 120, proximate a distal end of housing 110, and configured for connection to a complementary connector provided on or in the electrosurgical energy delivery device 30.

Housing 110 may be formed from any suitable material capable of housing transmission medium 120 and capable of providing sufficient strength for supporting the various components contained therewithin (e.g., connectors 112, 114). Housing 110 may include any number of sections or segments 110a joined to one another via interconnecting, articulatable joints 110b. Joints 110b may be any suitable hinge-like member capable of enabling deflection of one segment 110a of housing 110 relative to an adjacent segment 110a of housing 110. Joints 110b may be configured to enable adjacent sections 110a of housing 110 to deflect uni-directionally or omni-directionally.

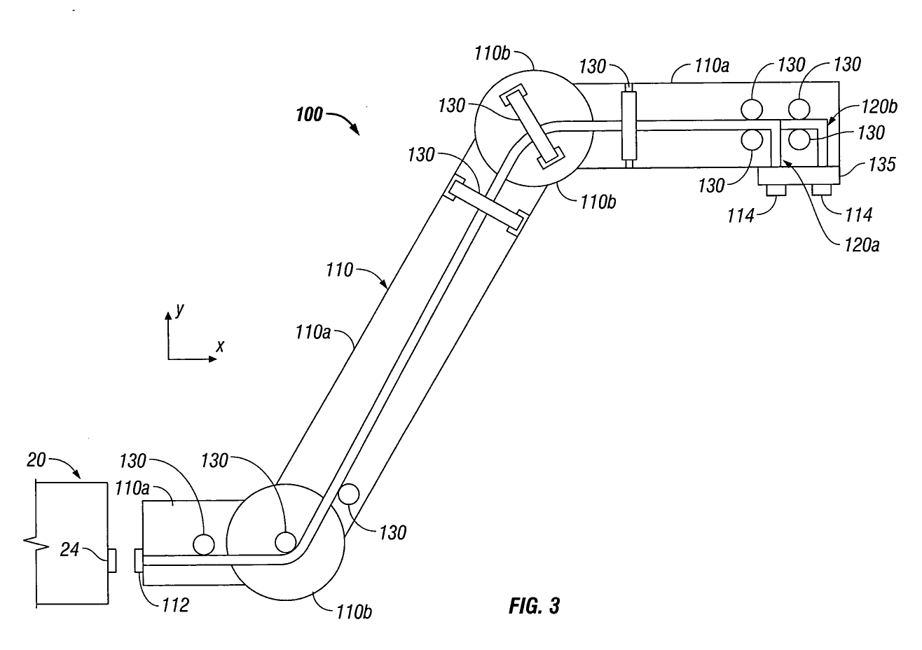

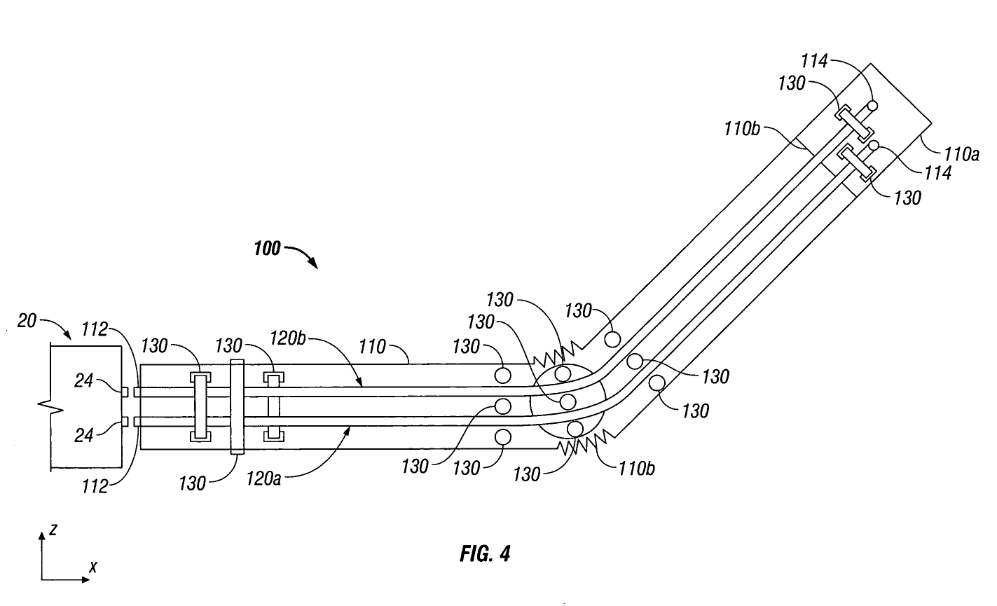

Housing 110 of delivery apparatus 100 may be configured to retain any number of transmission mediums or segments, such as, for example, two transmission mediums 120a, 120b, as shown in FIGS. 3 and 4.

Transmission medium 120 is formed from one or more sections of a relatively low loss cable (rigid, semi-rigid or flexible coaxial cable), a waveguide, a bus-bar or any other suitable means for transmitting electrosurgical energy or any desirable combination thereof. The various sections of transmission medium 120 may be coupled to one another using suitable low loss couplings or connectors (not shown). Suitable low loss couplings or connectors may include rotating couplings or connectors that insure that the physical limitations, e.g., minimum bend radiuses, operating tensions and temperatures, of the transmission medium are not violated. Exemplary transmission mediums 120 are shown and described in commonly owned U.S. application Ser. No. 11/85,052, filed on May 22, 2007, entitled “Energy Delivery Conduits for Use With Electrosurgical Devices”.

Suitable transmission media for use or inclusion in housing 110 of delivery apparatus 100 include, and are not limited to, a non-rigid/flexible coaxial cable (having a relatively high degree of transmission loss), a semi-rigid coaxial cable (having a relatively moderate-to-high degree of transmission loss), a fully rigid coaxial cable (having a relatively moderate degree of transmission loss), a fully rigid coaxial cable with an air dielectric (having a relatively moderate-to-low degree of transmission loss), and a wave-guide transmission medium (having a relatively low degree of transmission loss).

The particular type of transmission medium included in housing 110 will determine the type of joint or couplings interconnecting various sections of the transmission medium. For example, if a flexible coaxial cable is the selected transmission medium, no couplings may be required as a single length of said transmission medium is sufficiently flexible to pass through housing 110 and be relatively unaffected by joints 110b of housing 110.

If a transmission medium having a relatively higher degree of rigidity is selected, the transmission medium may be separated into individual segments corresponding to the various segments 110a of housing 110. In this embodiment, a suitable electromechanical coupling or fitting is employed to interconnect adjacent segments of the transmission medium. Electromechanical couplings are configured to permit adjacent segments of the transmission medium to pivot off axis relative to one another, to rotate axially relative to one another and to minimize energy loss from one segment of the transmission medium to the next.

As seen in FIG. 2A, delivery apparatus 100 is configured to selectively couple with various electrosurgical energy delivery devices 30, such as, for example, an electrosurgical pencil, probe, needle antenna or the like. Delivery device 30 may be directly coupled to second connector 114 of delivery apparatus 100, or delivery device 30 may be coupled to second connector 114 of delivery apparatus 100 via a further electrical conduit 32 or the like and a corresponding plug 34.

As seen in FIG. 2B, a connector expansion plug 125 may be provided for selective inter-connection with second connector 114. Expansion plug 125 is configured to expand the number of electrosurgical energy delivery devices 30 that may be coupled to delivery apparatus 110 via second connector 114. For example, as seen in FIG. 2B, expansion plug 125 may be configured to accommodate any suitable number of electrosurgical energy delivery devices 30 (three shown). Electrosurgical energy delivery devices 30 may be coupled to plug 125 via corresponding electrical conduits 32 and plugs 34. Alternatively, electrosurgical energy delivery devices 30 may be coupled to plug 125 via an articulating armature 32a and a corresponding plug 34.

With reference to FIGS. 3 and 4, the path of transmission medium 120 of delivery apparatus 100 may be defined by at least one guide or positioning member 130 that functions to guide and/or constrain transmission medium 120 along a path through sections 110a of housing 110. Guide members 130 aid in ensuring that the physical properties of transmission medium 120 are not violated as housing 110 is manipulated, for example, minimum bend radiuses and tensions. As shown in FIGS. 3 and 4, guide members 130 are configured to constrain transmission medium 120 and limit the movement and/or degree of deflection of transmission medium 120 to ensure that, as sections 110a of housing 110 are manipulated, any physical limitations of transmission medium 120 are not violated (e.g., minimum bend radius of transmission medium 120 that is a rigid or semi-rigid coaxial cable).

As seen in FIGS. 3 and 4, guide members 130 include pins, dowels or the like disposed on opposed sides of transmission medium 120. In this manner, as sections 110a of housing 110 are articulated with respect to one another about joints 110b, guide members 130 function to enlarge a radius of curvature of transmission medium 120 as transmission medium 120 is being bent. In so doing, the efficiency of the transmission of energy through a bent transmission medium 120 is maintained relatively high and/or the dissipation of energy through transmission medium 120 is minimized.

A position, orientation and/or location of delivery apparatus 100 may be achieved relative to, and independently of, the position of generator 20. For example, a distal-most end of housing 110 of delivery apparatus 100 may be moved in horizontal (X and Y axes) and vertical (Z axis) directions to optimize or maximize a distance between generator 20 and the distal-most end of housing 110. In particular, joints 110b of housing 110 are configured to enable movement of sections 110a of housing 110 is any direction and for maintaining the position of sections 110a of housing 110 after housing 110 is released by the user.

By way of example only, sections 110a of housing 110 may be interconnected by articulating and/or pivoting joints 110b which may have an adjustable tension or tightening feature enabling the joints to retain the relative positioning of sections 110a, to allow relatively free movement of any of sections 110a relative to one another, or allow relatively controlled movement of any of sections 110a relative to one another. It is contemplated that joints 110b may be selected such that when a distal end of housing 110 is unsupported or not held by the user, that housing 110 will remain in the last position moved to by the user.

Movement of segments 110a of housing 110 may be facilitated through one or more mechanical joints 110b, as mentioned above. Joints 110b may include hinge joints, ball joints or other suitable means to provide movement between two adjacent sections 110a. Joints 110b may be integrally formed with segments 110a of housing 110 or may be fixed to segments 110a of housing 110. In an alternate embodiment, at least one segment 110a of housing 110 may be formed of a semi-rigid, yet flexible, tube-like material that can be manipulated, flexed and/or bent in a plurality of positions and/or directions.

Since the distance between generator 20 and second connector 114 of electrosurgical energy delivery apparatus 100 is traversed by the low loss transmission medium 120, the dissipation and losses of energy therebetween are minimized. As such, a relatively more flexible medium, albeit higher energy dissipating, may be used to transfer power from second connector 114 of electrosurgical energy delivery apparatus 100 to a point of application or to an electrosurgical energy delivery device 30, without unduly increasing the dissipation and/or losses of energy of the entire system.

The electrosurgical energy delivery apparatus and the methods of use discussed above are not limited to microwave antennas used for hyperthermic, ablation, and coagulation treatments but may include any number of further microwave antenna applications. While several embodiments of the disclosure have been shown in the drawings and/or discussed herein, it is not intended that the disclosure be limited thereto, as it is intended that the disclosure be as broad in scope as the art will allow and that the specification be read likewise. Modification of the above-described system and methods for using the same, and variations of aspects of the disclosure that are obvious to those of skill in the art are intended to be within the scope of the claims. Therefore, the above description should not be construed as limiting, but merely as exemplifications of particular embodiments.

What is claimed is:

1. A delivery apparatus for supporting an electrosurgical instrument and for connecting the electrosurgical instrument to an electrosurgical energy source, the delivery apparatus comprising:
a selectively positionable housing including a plurality of rigid segments and a joint member interposed between each adjacent rigid member; and
a transmission medium extending through at least one of the segments of the housing, wherein the transmission medium is configured to transmit electrosurgical energy between the electrosurgical energy source and the electrosurgical instrument;
wherein the housing is movable between a first position wherein an electrosurgical instrument supported on an end of the housing is remote from an operative site and a second position wherein the electrosurgical instrument supported on the end of the housing is proximate to the operative site.

2. The delivery apparatus according to claim 1, wherein the transmission medium is continuous and flexible along at least a portion of its length.

3. The delivery apparatus according to claim 1, wherein the transmission medium includes a plurality of segments electromechanically joined to one another.

4. The delivery apparatus according to claim 3, wherein each segment of the housing supports at least one segment of the transmission medium.

5. The delivery apparatus according to claim 3, further comprising an electromechanical coupling inter-connecting adjacent segments of the transmission medium.

6. The delivery apparatus according to claim 5, wherein each electromechanical coupling is configured to permit adjacent segments of the transmission medium to at least one of pivot off axis relative to one another and rotate axially relative to one another.

7. The delivery apparatus according to claim 1, wherein the transmission medium is at least one of a relatively flexible coaxial cable, a semi-rigid coaxial cable, a fully rigid coaxial cable, a fully rigid coaxial cable with an air dielectric, and a wave-guide.

8. The delivery apparatus according to claim 3, wherein the housing prevents bending of the segments of the transmission medium along their individual lengths.

9. A system for maneuvering an electrosurgical instrument to/from an operative site and for connecting the electrosurgical instrument to an electrosurgical energy source, the system comprising:
an electrosurgical energy source;
an electrosurgical instrument; and
a delivery apparatus supported on and electrically coupled to the electrosurgical energy source, the delivery apparatus including:
a selectively positionable housing including a plurality of rigid segments and a joint member interposed between each adjacent rigid member, the housing being configured to support the electrosurgical instrument at a free end thereof; and
a transmission medium extending between the electrosurgical energy source and the electrosurgical instrument, through at least one of the segments of the housing, wherein the transmission medium is configured to transmit electrosurgical energy between the electrosurgical energy source and the electrosurgical instrument;
wherein the housing is movable between a first position wherein the electrosurgical instrument supported on the free end of the housing is remote from the operative site and a second position wherein the surgical instrument supported on the free end of the housing is proximate to the operative site.

10. The system according to claim 9, wherein the transmission medium is continuous and flexible along at least a portion of its length.

11. The system according to claim 9, wherein the transmission medium includes a plurality of segments electromechanically joined to one another.

12. The system according to claim 11, wherein each segment of the housing supports at least one segment of the transmission medium.

13. The system according to claim 11, wherein the delivery apparatus further includes an electromechanical coupling inter-connecting adjacent segments of the transmission medium.

14. The system according to claim 13, wherein each electromechanical coupling is configured to permit adjacent segments of the transmission medium to at least one of pivot off axis relative to one another and rotate axially relative to one another.

15. The system according to claim 9, wherein the transmission medium is at least one of a relatively flexible coaxial cable, a semi-rigid coaxial cable, a fully rigid coaxial cable, a fully rigid coaxial cable with an air dielectric, and a wave-guide.

16. The system according to claim 11, wherein the housing prevents bending of the segments of the transmission medium along their individual lengths.

17. The system according to claim 9, further comprising a connector coupled to the free end of the housing and electrically coupled to the transmission medium, the connector being configured to selectively connect a plurality of electrosurgical instruments to the electrosurgical energy source.

18. A method of maneuvering an electrosurgical instrument to/from a surgical site, wherein the electrosurgical instrument is electrically connected to an electrosurgical energy source, the method comprising the steps of:
providing a delivery apparatus supported on and electrically coupled to the electrosurgical energy source;
supporting a transmission medium on a housing of the delivery apparatus;
supporting the electrosurgical instrument on a free end of the housing and electrically connecting the electrosurgical instrument to the transmission medium;
manipulating the housing to move the electrosurgical instrument to a first position to perform an electrosurgical procedure; and
manipulating the housing to move the electrosurgical instrument to a second position upon completion of the electrosurgical procedure.

19. The method according to claim 18, further comprising the step of minimizing a loss of energy transmitted from the electrosurgical energy source to the electrosurgical instrument.

20. The method according to claim 18, further comprising the step of minimizing bending of the transmission medium between the electrosurgical energy source and the electrosurgical instrument.
